(19) United States
(12) Patent Application Publication
(10) Pub. No.: US 2026/0325569 A1
(43) Pub. Date: Oct. 22, 2026

(54) COMPOSITION FOR ENHANCING MOTIVATION

(71) Applicant: TAIYO KAGAKU Co., Ltd., Yokkaichi (JP)

(72) Inventors: Aya ABE, Yokkaichi-shi (JP); Makoto OZEKI, Yokkaichi-shi (JP)

(21) Appl. No.: 19/099,573

(22) PCT Filed: Jul. 27, 2023
(86) PCT No.: PCT/JP2023/027669

§ 371 Date: Jan. 29, 2025

(30) Foreign Application Priority Data
Aug. 8, 2022 (JP) 2022-126026

(51) Int. Cl. A61K 31/736; A61K 9/00; A61P 25/00

(52) U.S. Cl. CPC A61K 31/736; A61K 9/0053; A61P 25/00

(57) ABSTRACT

A method for increasing motivation after sleep or for improving wakefulness upon waking involves administering a composition that contains a partially hydrolyzed guar gum (PHGG), which has an average molecular weight of 1.0×10³ to 2.0×10⁵, with 70% or more by mass of its components falling within this range. A 1% by mass aqueous solution of the PHGG has a viscosity, measured at 25° C. and 60 rpm using a B-type viscometer, of 50 mPa·s or less. The PHGG may be produced by hydrolyzing galactomannan polysaccharide, which is obtained from guar endosperm and has a galactose to mannose content ratio (galactose:mannose) in the range of 1:1.3 to 1:2.1, using β-mannanase to reduce the molecular weight. The PHGG contains at least 70% by mass of dietary fiber as determined by an enzymatic HPLC method.

### CROSS-REFERENCE

The present application is the US national stage of International application serial no. PCT/JP2023/027669 filed on Jul. 27, 2023, which claims priority to Japanese patent application no. 2022-126026 filed on Aug. 8, 2022.

### TECHNICAL FIELD

The invention relates to compositions for increasing motivation and/or for improving wakefulness upon waking.

### BACKGROUND ART

People may have days in which they feel unmotivated due to aging, insufficient sleep, current mental state, and so on. Lack of motivation can lead to a decline in concentration and negative thinking. Research and development for improving such conditions is described in Patent Document 1. In addition to lack of motivation, people may also have difficulty waking up.

### PRIOR ART

Patent Document 1: JP 2022-097984A
Patent Document 2: JP 2006-096715A
Non Patent Document 1: Tanaka H, Shirakawa S, Sleep health, lifestyle and mental health in the Japanese elderly ensuring sleep to promote a healthy brain and mind. J Psychosomatic Research. 2004; 56:465-477
Non Patent Document 2: Maxwell C., Sensitivity and accuracy of the visual analogue scale: a psycho-physical classroom experiment. Br J Clin Pharmacol. 1978; 6:15-24.

### SUMMARY OF THE INVENTION

The composition described in Patent Document 1 noted above was a product that contains an extract and/or pulverized product of a Salacia plant. As it would be preferable to be able to provide more compositions that increase motivation, there is room for further research.

Therefore, it is one non-limiting object of the present teachings to provide a novel composition for increasing motivation and/or a composition for improving wakefulness upon waking, as well as treatment and preventive methods using such compositions.

The inventors have discovered that a composition containing a specific guar gum decomposition product has a motivation increasing effect.

Thus, one composition for increasing motivation after sleep according to one aspect of the present teachings comprises a partially hydrolyzed guar gum (PHGG), which has an average molecular weight of 1.0×10³ to 2.0×10⁵, with 70% or more by mass of the components thereof falling within this range. A 1% by mass aqueous solution of the PHGG has a viscosity, measured at 25° C. and 60 rpm using a B-type viscometer, of 50 mPa·s or less. The PHGG may be produced by hydrolyzing galactomannan polysaccharide, which is obtained from guar endosperm and has a galactose to mannose content ratio (galactose:mannose) in the range of 1:1.3 to 1:2.1, using β-mannanase to reduce the molecular weight. The PHGG contains at least 70% by mass of dietary fiber as determined by an enzymatic HPLC method.

In addition, one composition for improving wakefulness upon waking according to another aspect of the present teachings comprises PHGG, which has an average molecular weight of 1.0×10³ to 2.0×10⁵, with 70% or more by mass of the components thereof falling within this range. A 1% by mass aqueous solution of the PHGG has a viscosity, measured at 25° C. and 60 rpm, using a B-type viscometer, of 50 mPa·s or less. The PHGG may be produced by hydrolyzing galactomannan polysaccharide, which is obtained from guar endosperm and has a galactose to mannose content ratio (galactose:mannose) in the range of 1:1.3 to 1:2.1, using β-mannanase to reduce the molecular weight. The PHGG contains at least 70% by mass of dietary fiber as determined by an enzymatic HPLC method.

Such compositions for increasing motivation and/or for improving wakefulness is (are) preferably intended for healthy individuals that do not have a sleep disorder.

In another aspect of the present teachings, a food, drink or pharmaceutical product containing the composition for increasing motivation or the composition for improving awakening of the invention can be provided. Such food and drink products include, e.g., a product that includes food and beverages, such as nutritional supplements, health foods, foods for specified health uses, foods labelled as functional, foods for dietary therapy, general health foods, supplements, tea drinks, coffee drinks, juices, soft drinks, energy drinks, cooked rice, bread, noodles, dairy products, processed eggs, processed seafood and livestock foods, confectionery, oils and fats-processed foods, seasonings, and side dishes. Such pharmaceutical products include pharmaceutical products or quasi drug products, and are preferably oral preparations or enteral preparations, and can be in the form of liquids, tablets, granules, pills, syrups, and so on.

In short, a novel composition for increasing motivation and/or a novel composition for improving wakefulness upon waking can be provided.

### BRIEF DESCRIPTION OF THE DRAWINGS

FIG. 1 is a bar graph showing the results of the total sleep health risk score of PHGG.

FIG. 2 is a bar graph showing the results of an investigation of PHGG into the feeling of being refreshed upon waking.

FIG. 3 is a bar graph showing the results of an investigation of PHGG into the feeling of fatigue upon waking.

FIG. 4 is a bar graph showing the results of an investigation of PHGG into the motivation for work or study.

FIG. 5 is a bar graph showing the results of an investigation of PHGG into the vigor-vitality (VA) upon waking.

FIG. 6 is a bar graph showing the results of an investigation of PHGG into the sleepiness upon waking.

### DETAILED DESCRIPTION OF EMBODIMENTS OF THE PRESENT INVENTION

Next, embodiments of the present teachings will be described with reference to tables and drawings. The technical scope of the invention is not limited to these embodiments, and the invention can be embodied in various forms without changing the gist of the invention.

“Guar gum” refers to a water-soluble natural polysaccharide obtained from the endosperm of guar beans (specifically, cotyledons). Such a polysaccharide has a side chain of one galactose molecule attached to two linearly bonded mannose molecules, and has an average molecular weight of about 2.0×10⁵ to 3.0×10⁵. Guar gum is known to have physiological effects such as suppressing an increase in blood sugar level, lowering cholesterol, and improving bowel movements. In the present disclosure, the term “partially hydrolyzed guar gum” refers to a water-soluble dietary fiber obtained by hydrolyzing and thereby decreasing the molecular weight of the galactomannan polysaccharide contained in the endosperm of guar (scientific name: Cyanopsis tetragonoloba), an annual legume plant native to India, Pakistan, etc., which is used as a raw material. Methods for hydrolyzing guar gum are not particularly limited, and may include enzymatic decomposition, acidic decomposition, or the like. Enzymatic decomposition is preferable because it is easy to adjust the molecular weight of the decomposition (hydrolyzed) products.

Although the enzyme(s) used in enzymatic hydrolysis is (are) not particularly limited as long as the enzyme(s) hydrolyze(s) a linear mannose chain, it is preferable to use β-mannanase derived from Aspergillus or Rhizopus. The upper limit of the average molecular weight distribution of the partially hydrolyzed guar gum is 2.0×10⁵ or less, preferably 1.0×10⁵ or less, and more preferably 2.5×10⁴ or less. The lower limit of the average molecular weight distribution of the partially hydrolyzed guar gum is 1.0×10² or more, preferably 2.0×10³ or more. If the average molecular weight exceeds 2.0×10⁵, the viscosity becomes too high and it becomes difficult to integrate the partially hydrolyzed guar gum into foods and drinks. Although the methods for measuring the molecular weight distribution are not particularly limited, suitable methods include, for example, gel filtration chromatography that uses polyethylene glycols (average molecular weights: 2×10², 2×10³, 2×10⁴ and 1×10⁵) as molecular weight markers.

With regard to the partially hydrolyzed guar gum (PHGG) according to the present teachings, products that contain at least 70% by mass, preferably at least 80% by mass, of components falling within the above-mentioned average molecular weight range can be used.

Galactose is a type of monosaccharide classified as an aldohexose; the molecular formula is C₆H₁₂O₆ and the molecular weight is 180 g/mol (both are the same as glucose). With regard to the molecular configuration, the hydroxyl groups (—OH) at the 2nd position (second from the top in the Fischer projection) and 5th position point (extend) in the same direction, while those at the 3rd and 4th positions are in the opposite direction, and the structure at the 5th position of D-galactose is the same as that of D-glyceraldehyde.

Mannose is a type of monosaccharide that is also classified as an aldohexose; the molecular formula is also C₆H₁₂O₆ and the molecular weight is also 180 (both are the same as glucose). With regard to the molecular configuration, the hydroxyl groups (—OH) at the 2nd and 3rd positions point (extend) in the same direction, while those at the 4th and 5th positions are in the opposite direction, and the structure at the 5th position of D-mannose is the same as that of D-glyceraldehyde. Mannose is not metabolized by humans, and when ingested orally, very little of the mannose enters the glycolytic pathway.

“Motivation” is an expression that refers to a positive feeling or desire to do or accomplish things on one's own. It is recognized that motivation is brought about by dopamine secreted from the brain. It is known that motivation can be lost due to irregular lifestyle habits, accumulated fatigue, lack of sleep, and so on.

The compositions for increasing motivation of the present teachings can be taken orally as is or mixed with food or beverages, and so on. Although the dosage of the composition for increasing motivation when taken orally is not particularly limited, it is from 0.5 g to 70 g (preferably 3 g to 30 g, more preferably 6 g to 18 g) per adult per day.

### Preparation of Partially Hydrolyzed Guar Gum (PHGG)

### Example 1

After adjusting the pH to 4.5 by adding 0.1N hydrochloric acid to 900 g of water, 0.2 g of commercially available β-mannanase derived from Aspergillus bacteria and 100 g of guar gum powder were added and mixed. This mixture was reacted at 40° C. to 45° C. for 24 hours. After the reaction, the enzyme was inactivated by heating at 90° C. for 15 minutes. The reaction solution was filtered and separated by suction filtration, and the transparent solution obtained by removing insoluble matter was concentrated under reduced pressure (an evaporator manufactured by Yamato). A solid content of 20% by mass was obtained. This was dried using a spray dryer (OHKAWARA KAKOHKI Co., Ltd.), yielding 65 g of partially hydrolyzed guar gum (PHGG) as a powder.

The partially hydrolyzed guar gum was dissolved in water to prepare an aqueous solution having a concentration of 0.5% (w/v). The average molecular weight was determined by gel filtration chromatography (column: YMC-Pack Diol-120, detector: differential refractometer) using polyethylene glycol (average molecular weights: 2×10², 2×10³, 2×10⁴ and 1×10⁵) as molecular weight markers, and was found to be approximately 20,000, with more than 80% by mass having molecular weights in the range of 1.0×10³ to 2.0×10⁵.

Furthermore, the viscosity of a 1% by mass aqueous solution of the PHGG was measured using a B-type viscometer at 25° C. and 60 rpm, and was found to be 8 mPa·s.

The galactose to mannose content ratio (galactose:mannose) was measured, and was found to be 1:1.7.

The dietary fiber content was measured by enzymatic HPLC and was found to be 90% by mass.

### Example 2

After adjusting the pH to 3 by adding 0.1N hydrochloric acid to 900 g of water, 0.15g of commercially available β-mannanase derived from Aspergillus bacteria and 100 g of guar gum powder were added and mixed. This mixture was reacted at 40° C. to 45° C. for 24 hours. After the reaction, the enzyme was inactivated by heating at 90° C. for 15 minutes. The reaction solution was filtered and separated by suction filtration, and the transparent solution obtained by removing insoluble matter was concentrated under reduced pressure (an evaporator manufactured by Yamato). A solid content of 20% by mass was obtained. This was dried using a spray dryer (OHKAWARA KAKOHKI Co., Ltd.), yielding 68 g of partially hydrolyzed guar gum as a powder.

The average molecular weight of the PHGG was determined in the same manner as in Example 1 and was approximately 2.5×10⁴. The HPLC chart showed that the molecular weight was 1.0×10³ to 2.0×10⁴ for 80% by mass or more of the components of the PHGG.

Furthermore, the viscosity of a 1% by mass aqueous solution was measured using a B-type viscometer at 25° C. and 60 rpm, and was found to be 10 mPa·s.

The galactose to mannose content ratio (galactose:mannose) was measured, and was found to be 1:1.8.

The dietary fiber content was measured using an enzymatic HPLC method, and was found to be 89% by mass.

### Example 3

After adjusting the pH to 4 by adding 0.1N hydrochloric acid to 900 g of water, 0.25 g of commercially available β-mannanase derived from Aspergillus bacteria and 100 g of guar gum powder were added and mixed. This mixture was reacted at 50° C. to 55° C. for 12 hours. After the reaction, the enzyme was inactivated by heating at 90° C. for 15 minutes. The reaction solution was filtered and separated by suction filtration, and the transparent solution obtained by removing insoluble matter was concentrated under reduced pressure (Yamato evaporator). A solid content of 20% by mass was obtained. This was dried using a spray dryer (OHKAWARA KAKOHKI Co., Ltd.), yielding 70 g of PHGG as a powder.

The average molecular weight of the PHGG was determined in the same manner as in Example 1 and was approximately 1.5×10⁴. The HPLC chart showed that 80% by mass or more of the components of the PHGG had a molecular weight in the range from 1.0×10³ to 2.0×10⁵.

Furthermore, the viscosity of a 1% by mass aqueous solution was measured using a B-type viscometer at 25° C. and 60 rpm, and was found to be 9 mPa·s.

The galactose to mannose content ratio (galactose:mannose) was measured, and was found to be 1:2.0.

The dietary fiber content was measured using an enzymatic HPLC method, and was found to be 88% by mass.

### Example 4

PHGG was prepared according to the description of an example (page 4, line 3 to page, 4, line 10) in JP H05-117156 A. The average molecular weight was determined according to Example 1, and was found to be 5.5×10³.

### Test Method 1

A placebo-controlled, double-blind, randomized, parallel-group study was conducted to evaluate the effects of PHGG on feeling and motivation upon waking up. Sixty healthy subjects aged 30 to 50 years were divided into three groups of 20 subjects each, and for 8 weeks the groups respectively ingested placebo powdered food, 3 g PHGG of Example 1 per day, and 5 g PHGG of Example 1 per day.

Questionnaire surveys on sleep and motivation were administered at weeks 0, 4, and 8, and the total sleep health risk score and scores for feeling refreshed upon awakening, feeling tired upon awakening, and feeling motivated to work or study were calculated.

“Sleep Health Risk Index total score (SHRI total score)” was calculated from the sum of the scores for each evaluation item of the Sleep Health Risk Index (SHRI) (Non-Patent Document 1), including the following factors: Factor 1 (sleep maintenance disorders), Factor 2 (parasomnia-related symptoms), Factor 3 (sleep apnea), Factor 4 (difficulty waking up), and Factor 5 (sleep onset disorders). A higher score indicates a higher risk level for overall sleep health.

Scores for feeling and motivation upon waking were scored from 0 (worst) to 10 (best) using the visual analog scale (VAS) (Non-Patent Document 2). Higher scores indicate better health.

### Test Results 1

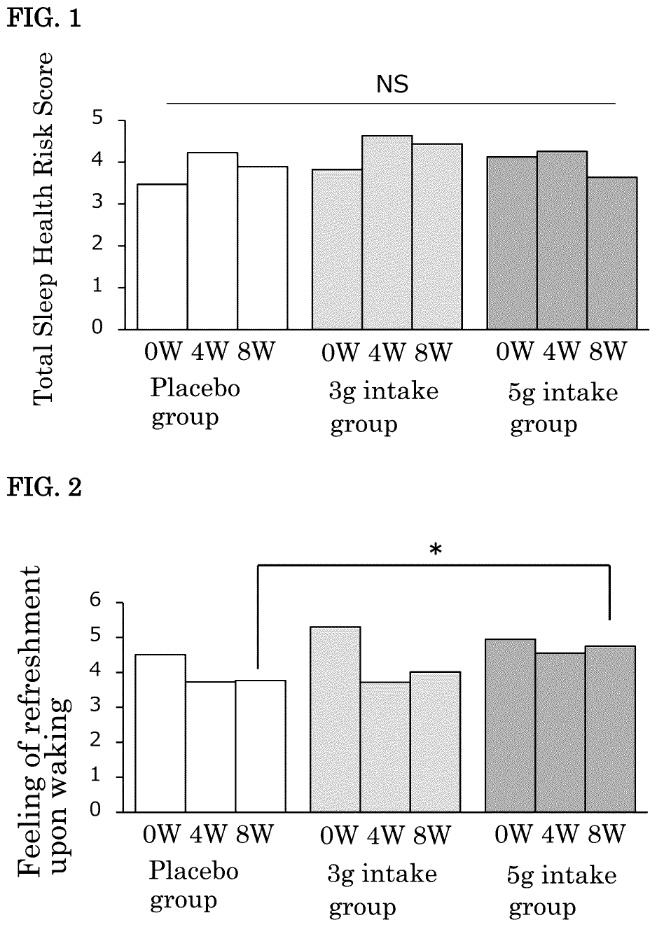

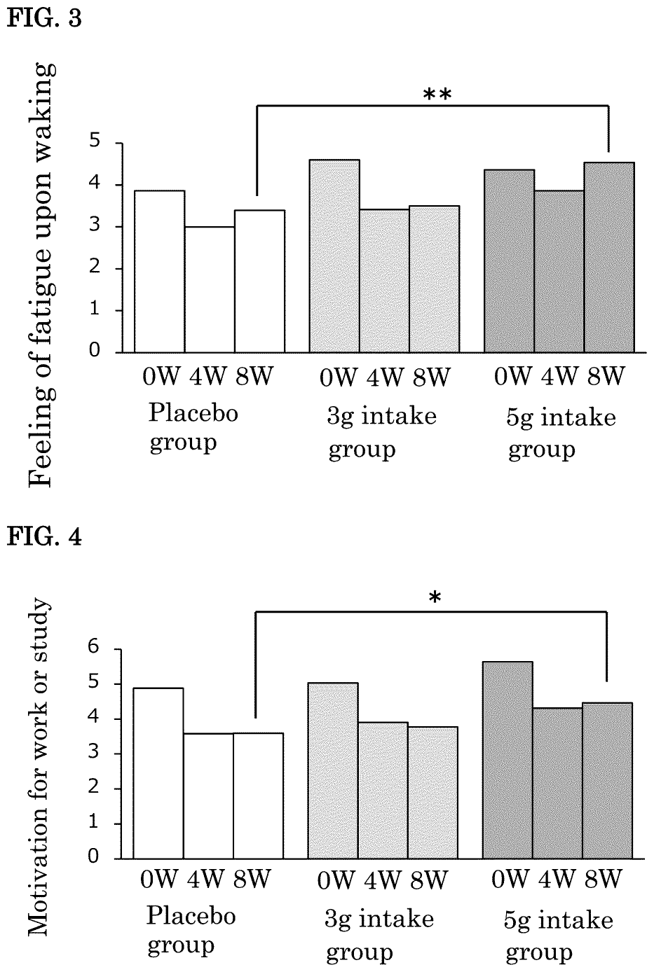

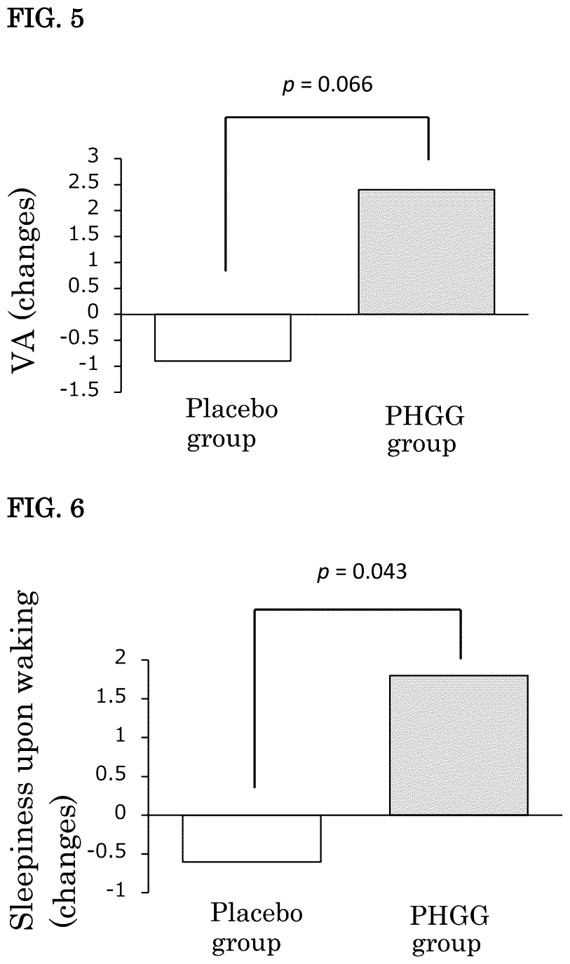

The total scores for sleep health risk are shown in Table 1 and FIG. 1, while the results of the VAS questionnaire concerning the feeling of being refreshed upon waking, the feeling of being fatigued upon waking, and motivation for work or study, respectively, are shown in Table 2 and FIGS. 2 to 4. It is noted that, because there were dropouts in each group, data analysis was performed on 19 cases in the placebo group, 14 cases in the PHGG (3 g intake group), and 17 cases in the PHGG (5 g intake group).

TABLE 1

| ITEM | Group | 0 W | 4 W | 8 W |
|---|---|---|---|---|
| Total Sleep | Placebo group | 3.47 ± 1.59 | 4.23 ± 1.16 | 3.89 ± 0.94 |
| Health Risk | 3 g intake | 3.82 ± 1.72 | 4.63 ± 1.44 | 4.44 ± 2.27 |
| Score | group | | | |
| | 5 g intake | 4.12 ± 3.06 | 4.26 ± 1.87 | 3.64 ± 0.86 |
| | group | | | |

TABLE 2

| ITEM | Group | 0 W | 4 W | 8 W |
|---|---|---|---|---|
| Feeling of | Placebo group | 4.51 ± 2.38 | 3.73 ± 1.33 | 3.77 ± 1.14 |
| refreshment | 3 g intake group | 5.30 ± 2.37 | 3.71 ± 1.36 ^{##} | 4.01 ± 1.35 ^{#} |
| upon waking | 5 g intake group | 4.95 ± 2.71 | 4.55 ± 1.25 | 4.76 ± 1.21 * |
| Feeling of fatigue | Placebo group | 3.86 ± 2.22 | 3.00 ± 1.29 | 3.39 ± 1.16 |
| upon waking | 3 g intake group | 4.60 ± 2.03 | 3.41 ± 1.37 ^{#} | 3.50 ± 0.94 |
| | 5 g intake group | 4.36 ± 2.55 | 3.86 ± 1.26 | 4.54 ± 1.30 ** |
| Motivation for | Placebo group | 4.88 ± 2.51 | 3.58 ± 1.27 ^{#} | 3.60 ± 1.10 |
| work or study | 3 g intake group | 5.04 ± 1.72 | 3.91 ± 1.06 ^{#} | 3.78 ± 1.11 ^{##} |
| | 5 g intake group | 5.64 ± 1.88 | 4.31 ± 1.09 ^{##} | 4.46 ± 0.88 *^{, ##} |

average ± S.D.
comparison with placebo group:
** p < 0.01,
* p < 0.05
within-group comparison:
^{##} p < 0.01,
^{#} p < 0.05

In Patent Document 2, it was disclosed that dietary fiber has an effect on improving sleep health. According to the test described in Patent Document 2, it was shown that cookies containing dietary fiber (gum arabic) improved the total sleep health risk score (paragraph 0021). Based on this disclosed technology, the inventors conducted a test on dietary fiber containing a specific low molecular weight guar gum, hoping that it would have a sleep improving effect. However, surprisingly, for the above-described composition, no significant difference was observed in the sleep health risk total score between the placebo group and the PHGG groups (both 3 g and 5 g) (Table 1 and FIG. 1). For this reason, it was necessary to give up using the PHGG of the present teachings for comprehensive sleep improvement.

However, when the effects of the PHGG of the present teachings were reviewed from a different perspective, a favorable effect was observed. That is, significant differences were observed between the placebo group and the PHGG group in terms of the feeling of being refreshed upon waking up, the feeling of fatigue upon waking up, and the motivation for work or study (Table 2 and FIGS. 2 to 4). It is noted that the PHGGs of Examples 2 to 4 were also examined, and similar effects were observed. Thus, it was found that, with regard to specific PHGGs, although there was no overall sleep health improving effect, there were effects on increasing motivation and/or improving wakefulness upon waking up. In particular, this is new in that it has an effect of increasing motivation after sleep and/or improving wakefulness upon waking up in healthy subjects who do not have a sleep disorder.

### Test Results 2

A placebo-controlled, double-blind, randomized, parallel-group comparative study was conducted to verify the effects of PHGG on sleepiness upon waking, sleep onset and maintenance, dreaming, fatigue recovery, and sleep time in healthy elderly subjects. Healthy male and female subjects age 60 years or older were included.

For the 91 screened cases, 66 healthy subjects who were not included in the exclusion criteria were included. Exclusion criteria included people having a history of malignant tumors or heart disease; people having pacemakers; patients treated for arrhythmia, hepatic disorders, renal disorders, diabetes, dyslipidemia, hypertension, and so on; regular users of medicines or supplements; those with allergies; people having exercise habits (more than 2 times/week, 30 minutes/time, 1 year); people infected with the new corona virus; people who participated in other clinical trials within 28 days prior to the date of obtaining the consent form; and other subjects deemed inappropriate by the responsible physician.

Sixty six subjects were randomly divided into two groups, one of which was the test food group (33 subjects) and the other was the placebo group (33 subjects). The subjects of the test food group took 5 g/day of PHGG from Example 1, and the subjects of the placebo group took 5 g/day of placebo powdered food every day with breakfast for 12 weeks. It is noted that, because 3 subjects in the subject group and 2 subjects in the placebo group dropped out during the study, data from 30 subjects and 31 subjects, respectively, were analyzed.

Statistical analysis was performed on the scores of POMS2 and OSA-MA at weeks 0, 8, and 12. POMS2 is a test that can quickly evaluate “not only relatively long-lasting emotional states, but also fluctuating, transient emotions”. It includes seven factors: “Anger-Hostility (AH)”, “Confusion-Bewilderment (CB)”, “Depression-Dejection (DD)”, “Fatigue-Inertia (FI)”, “Tension-Anxiety (TA)”, “Vigor-Activity (VA)”, and “Friendliness (F)”, and includes TMD as an indicator of overall mood state. A score below 30 corresponds to “very low”, 30-39 to “low”, 40-59 to “average”, 60-69 to “high”, and a score above 70 to “very high”; for TMD, AH, CB, DD, FI, and TA, the lower the score, the better the condition, whereas for VA and F, the higher the score, the better the condition.

OSA-MA is a test that evaluates sleepiness upon waking, sleep onset and maintenance, dreaming, fatigue recovery, and sleep duration, and the test was performed at home upon waking on each of the three days prior to the test (the day of the test, the day before, and the day before that). The average of the three days was used as the measured score.

The POMS2 score data are shown in Tables 3 and 4. Although no significant differences were confirmed between two groups in the POMS2 scores, the actual measured score of VA and the change from baseline after 8 weeks of intake tended to be higher in the test food group than the placebo group (both P=0.066), suggesting that intake of PHGG improves vitality and energy.

TABLE 3

| | | | | | | | | | | | | | | | | within [symbol] group comparison | | | | |
|---|---|---|---|---|---|---|---|---|---|---|---|---|---|---|---|---|---|---|---|---|
| | | | | test food group | | | | | | placebo group | | | | | | | | 95% | 95% | |
| ITEM | UNIT | TIME | | n | Mean | SD | Med | Min | Max | n | Mean | SD | Med | Min | Max | [symbol] | SE | CI− | CI+ | P |
| TMD | score | VISIT1 | * | 30 | 42.9 | 6.7 | 42.0 | 31.0 | 63.0 | 31 | 42.4 | 6.3 | 43.0 | 30.0 | 54.0 | 0.5 | 1.7 | −2.8 | 3.8 | 0.759 |
| | | VISIT2 | # | 30 | 42.1 | 6.1 | 41.5 | 29.0 | 53.0 | 31 | 42.8 | 7.1 | 43.0 | 32.0 | 58.0 | −1.1 | 0.9 | −2.9 | 0.7 | 0.235 |
| | | VISIT3 | # | 30 | 42.0 | 7.1 | 43.0 | 31.0 | 57.0 | 31 | 42.9 | 7.1 | 43.0 | 31.0 | 59.0 | −1.4 | 0.9 | −3.2 | 0.5 | 0.140 |
| | | Change1 | # | 30 | −0.7 | 3.4 | −1.0 | −8.0 | 6.0 | 31 | 0.4 | 3.6 | 0.0 | −6.0 | 14.0 | −1.1 | 0.9 | −2.9 | 0.7 | 0.235 |
| | | Change2 | # | 30 | −0.9 | 4.0 | −1.0 | −9.0 | 8.0 | 31 | 0.5 | 3.2 | 0.0 | −6.0 | 8.0 | −1.4 | 0.9 | −3.2 | 0.5 | 0.140 |
| AH | score | VISIT1 | * | 30 | 43.0 | 4.7 | 44.0 | 38.0 | 52.0 | 31 | 44.3 | 6.0 | 43.0 | 38.0 | 62.0 | −1.3 | 1.4 | −4.1 | 1.5 | 0.354 |
| | | VISIT2 | # | 30 | 43.2 | 4.7 | 43.5 | 38.0 | 52.0 | 31 | 44.4 | 6.7 | 41.0 | 38.0 | 63.0 | −0.2 | 1.1 | −2.3 | 1.9 | 0.842 |
| | | VISIT3 | # | 30 | 42.6 | 5.9 | 40.5 | 38.0 | 65.0 | 31 | 44.7 | 6.7 | 43.0 | 38.0 | 66.0 | −1.0 | 1.1 | −3.1 | 1.2 | 0.371 |
| | | Change1 | # | 30 | 0.1 | 3.3 | 0.0 | −11.0 | 10.0 | 31 | 0.1 | 4.5 | 0.0 | −10.0 | 14.0 | −0.2 | 1.1 | −2.3 | 1.9 | 0.842 |
| | | Change2 | # | 30 | −0.4 | 4.6 | 0.0 | −10.0 | 14.0 | 31 | 0.4 | 4.2 | 0.0 | −10.0 | 15.0 | −1.0 | 1.1 | −3.1 | 1.2 | 0.371 |
| CB | score | VISIT1 | * | 30 | 47.2 | 8.4 | 46.0 | 30.0 | 68.0 | 31 | 45.5 | 7.3 | 47.0 | 33.0 | 59.0 | 1.7 | 2.0 | −2.4 | 5.7 | 0.408 |
| | | VISIT2 | # | 30 | 45.4 | 7.1 | 46.0 | 32.0 | 59.0 | 31 | 45.7 | 8.0 | 47.0 | 33.0 | 65.0 | −1.6 | 1.2 | −4.0 | 0.8 | 0.181 |
| | | VISIT3 | # | 30 | 44.9 | 7.9 | 44.5 | 33.0 | 59.0 | 31 | 45.2 | 7.7 | 45.0 | 30.0 | 58.0 | −1.6 | 1.2 | −4.0 | 0.7 | 0.173 |
| | | Change1 | # | 30 | −1.8 | 5.2 | −2.5 | −11.0 | 11.0 | 31 | 0.2 | 5.1 | 0.0 | −8.0 | 17.0 | −1.6 | 1.2 | −4.0 | 0.8 | 0.181 |
| | | Change2 | # | 30 | −2.2 | 5.1 | −2.5 | −12.0 | 11.0 | 31 | −0.3 | 4.2 | −1.0 | −7.0 | 8.0 | −1.6 | 1.2 | −4.0 | 0.7 | 0.173 |
| DD | score | VISIT1 | * | 30 | 45.0 | 4.3 | 44.0 | 40.0 | 58.0 | 31 | 45.6 | 5.3 | 43.0 | 40.0 | 56.0 | −0.6 | 1.2 | −3.1 | 1.8 | 0.604 |
| | | VISIT2 | # | 30 | 44.8 | 4.1 | 44.0 | 40.0 | 54.0 | 31 | 45.3 | 5.1 | 44.0 | 40.0 | 55.0 | 0.0 | 0.8 | −1.5 | 1.5 | 0.970 |
| | | VISIT3 | # | 30 | 44.5 | 4.5 | 43.5 | 40.0 | 55.0 | 31 | 45.7 | 5.2 | 44.0 | 40.0 | 62.0 | −0.7 | 0.8 | −2.2 | 0.8 | 0.373 |
| | | Change1 | # | 30 | −0.2 | 3.6 | 0.0 | −7.0 | 11.0 | 31 | −0.4 | 3.0 | −1.0 | −7.0 | 10.0 | 0.0 | 0.8 | −1.5 | 1.5 | 0.970 |
| | | Change2 | # | 30 | −0.5 | 3.1 | −0.5 | −9.0 | 6.0 | 31 | 0.0 | 2.9 | 0.0 | −8.0 | 10.0 | −0.7 | 0.8 | −2.2 | 0.8 | 0.373 |

[symbol]

indicates data missing or illegible when filed

TABLE 4

| | | | | | | | | | | | | | | | | within [symbol] group comparison | | | | |
|---|---|---|---|---|---|---|---|---|---|---|---|---|---|---|---|---|---|---|---|---|
| | | | | test food group | | | | | | placebo group | | | | | | | | 95% | 95% | |
| ITEM | UNIT | TIME | | n | Mean | SD | Med | Min | Max | n | Mean | SD | Med | Min | Max | [symbol] | SE | CI− | CI+ | P |
| F [symbol] | score | VISIT1 | * | 30 | 42.3 | 7.9 | 41.0 | 34.0 | 61.0 | 31 | 40.6 | 4.9 | 41.0 | 34.0 | 48.0 | 1.7 | 1.7 | −1.7 | 5.1 | 0.322 |
| | | VISIT2 | # | 30 | 42.4 | 6.2 | 41.0 | 34.0 | 61.0 | 31 | 41.5 | 6.7 | 41.0 | 34.0 | 59.0 | −0.3 | 1.2 | −2.7 | 2.1 | 0.815 |
| | | VISIT3 | # | 30 | 41.5 | 7.8 | 38.0 | 34.0 | 62.0 | 31 | 41.9 | 6.6 | 41.0 | 34.0 | 57.0 | −1.8 | 1.2 | −4.2 | 0.6 | 0.139 |
| | | Change1 | # | 30 | 0.1 | 5.1 | 0.0 | −15.0 | 11.0 | 31 | 0.9 | 5.3 | 0.0 | −11.0 | 20.0 | −0.3 | 1.2 | −2.7 | 2.1 | 0.815 |
| | | Change2 | # | 30 | −0.8 | 4.6 | 0.0 | −13.0 | 7.0 | 31 | 1.3 | 4.9 | 0.0 | −7.0 | 18.0 | −1.8 | 1.2 | −4.2 | 0.6 | 0.139 |
| TA | score | VISIT1 | * | 30 | 43.6 | 7.2 | 43.0 | 32.0 | 63.0 | 31 | 44.2 | 6.1 | 46.0 | 33.0 | 54.0 | −0.6 | 1.7 | −4.0 | 2.8 | 0.730 |
| | | VISIT2 | # | 30 | 44.1 | 7.3 | 44.5 | 33.0 | 58.0 | 31 | 44.7 | 6.8 | 45.0 | 32.0 | 58.0 | −0.1 | 1.2 | −2.5 | 2.3 | 0.919 |
| | | VISIT3 | # | 30 | 43.9 | 7.8 | 44.5 | 32.0 | 66.0 | 31 | 45.0 | 7.9 | 45.0 | 33.0 | 59.0 | −0.6 | 1.2 | −2.9 | 1.8 | 0.647 |
| | | Change1 | # | 30 | 0.4 | 4.9 | 0.0 | −9.0 | 13.0 | 31 | 0.5 | 4.3 | 1.0 | −8.0 | 12.0 | −0.1 | 1.2 | −2.5 | [symbol] | 0.919 |
| | | Change2 | # | 30 | 0.2 | 5.2 | 0.5 | −9.0 | 13.0 | 31 | 0.7 | 4. [symbol] | 0.0 | −6.0 | 11.0 | −0.6 | 1.2 | −2.9 | 1.8 | 0.647 |
| VA | score | VISIT1 | * | 30 | 55.4 | 11.0 | 55.0 | 38.0 | 78.0 | 31 | 57.8 | 10.2 | 56.0 | 41.0 | 83.0 | −2.5 | 2.7 | [symbol] | 3.0 | 0.366 |
| | | VISIT2 | # | 30 | 57.8 | 9.9 | 55.5 | 42.0 | 83.0 | 31 | 57.0 | 9.7 | 58.0 | 40.0 | 80.0 | 2.7 | 1.4 | [symbol] | 5.6 | 0.066 |
| | | VISIT3 | # | 30 | 56.0 | 10.5 | 57.0 | 35.0 | 74.0 | 31 | 57.1 | 9.1 | 56.0 | 40.0 | 81.0 | 0.8 | 1.4 | −2.1 | [symbol] | 0.575 |
| | | Change1 | # | 30 | 2.4 | 6.2 | 2.5 | −9.0 | 18.0 | 31 | −0.9 | 6.4 | 0.0 | −11.0 | 21.0 | 2.7 | 1.4 | −0.2 | 5.6 | 0.066 |
| | | Change2 | # | 30 | 0.6 | 6.5 | 1.0 | −10.0 | 15.0 | 31 | −0.8 | 5.3 | −2.0 | −10.0 | 14.0 | 0.8 | 1.4 | −2.1 | 3.7 | 0.575 |
| F | score | VISIT1 | * | 30 | 57.6 | 9.3 | 54.0 | 42.0 | 75.0 | 31 | 59.4 | 8.4 | 59.0 | 44.0 | 75.0 | −1.8 | 2.3 | −6.4 | 2.7 | 0.427 |
| | | VISIT2 | # | 30 | 57.9 | 8.4 | 56.0 | 42.0 | 73.0 | 31 | 59.6 | 9.5 | 59.0 | 42.0 | 75.0 | −0.4 | 1.7 | −3.8 | 3.1 | 0.829 |
| | | VISIT3 | # | 30 | 57.6 | 10.0 | 55.0 | 37.0 | 75.0 | 31 | 58.9 | 8.6 | 59.0 | 44.0 | 78.0 | 0.0 | 1.7 | −3.5 | 3.4 | 0.983 |
| | | Change1 | # | 30 | 0.3 | 6.6 | 0.0 | −12.0 | 19.0 | 31 | 0.2 | 7.0 | 0.0 | −16.0 | 20.0 | −0.4 | 1.7 | −3.8 | 3.1 | 0.829 |
| | | Change2 | # | 30 | 0.1 | 8.4 | 2.0 | −26.0 | 14.0 | 31 | −0.5 | 6. [symbol] | 0.0 | −15.0 | 14.0 | 0.0 | 1.7 | −3.5 | 3.4 | 0.983 |

[symbol]

indicates data missing or illegible when filed

The average (Mean), standard deviation (SD), median (Med), minimum (Min), maximum (Max), between-group difference (Δ) and standard error (SE), minimum 95% confidence interval (95% CI−), maximum 95% confidence interval (95% CI+), and statistical analysis results for each OSA-MA score are shown in Table 5.

OSA-MA is a psychological scale that assesses sleep phases upon waking, and a higher score indicates a better state. From among the various scores of the OSA-MA, the effects on “sleepiness upon waking” by ingesting partially hydrolyzed guar gum were confirmed. The actual scores and changes from the baseline after 8 weeks of ingestion were significantly higher in the test food group than in the placebo group, and the actual values and changes from the baseline after 12 weeks of ingestion tended to be higher in the test food group than in the placebo group (both P=0.096). The actual score after 8 weeks was (test food group: 20.1±4.4 points, placebo group: 19.6±6.0 points, difference between groups: 1.8 points [0.1, 3.6], P=0.043), the change 1 after 8 weeks was (test food group: 1.8±3.6 points, placebo group: −0.6±3.9 points, difference between groups: 1.8 points [0.1, 3.6], P=0.043), the actual score after 12 weeks was (test food group: 20.0±4.2 points, placebo group: 19.9±6.6 points, difference between groups: 1.5 points [−0.3, 3.3], P=0.096), and the change 2 after 12 weeks was (test food group: 1.7±3.5 points, placebo group: −0.3±4.1 points, between-group difference: 1.5 points [−0.3, 3.3], P=0.096).

On the other hand, no differences between the groups were observed in other measures concerning sleep.

In this way, improved alertness upon waking was observed in healthy elderly subjects age 60 years or older, just as in healthy subjects aged 30 years or older and younger than 50 years.

TABLE 5

| | | | | | | | | | | | | | | | | within [symbol] group comparison | | | | |
|---|---|---|---|---|---|---|---|---|---|---|---|---|---|---|---|---|---|---|---|---|
| | | | | test food group | | | | | | placebo group | | | | | | | | 95% | 95% | |
| ITEM | UNIT | TIME | | n | Mean | SD | Med | Min | Max | n | Mean | SD | Med | Min | Max | [symbol] | SE | CI− | CI+ | P |
| [symbol] | score | VISIT1 | * | 30 | 18.4 | 4.6 | 18.8 | 10.8 | 32.3 | 31 | 20.2 | 6.7 | 20.3 | 6.0 | 32.3 | −1.9 | 1.5 | −4.8 | 1.1 | 0.204 |
| | | VISIT2 | # | 30 | 20.1 | 4.4 | 20.3 | 11.5 | 32.3 | 31 | 19.6 | 6.0 | 18.8 | 11.5 | 32.3 | 1.8 | 0.9 | 0.1 | 3.6 | 0.043 |
| | | VISIT3 | # | 30 | 20.0 | 4.2 | 20.3 | 10.8 | 31.3 | 31 | 19.9 | 6.6 | 20.3 | 10.5 | 32.3 | 1.5 | 0.9 | −0.3 | 3.3 | 0.096 |
| | | Change1 | # | 30 | 1.8 | 3.6 | 1.2 | −5.2 | 9.3 | 31 | −0.6 | 3.9 | 0.0 | −14.3 | 7.3 | 1.8 | 0.9 | 0.1 | 3.6 | 0.043 |
| | | Change2 | # | 30 | 1.7 | 3.5 | 1.1 | −5.7 | 13.0 | 31 | −0.3 | 4.1 | 0.0 | −9.5 | 10.8 | 1.5 | 0.9 | −0.3 | 3.3 | 0.096 |
| [symbol] | score | VISIT1 | * | 30 | 17.0 | 5.3 | 16.5 | 8.4 | 29.6 | 31 | 17.9 | 6.0 | 17.4 | 3.2 | 29.6 | −0.9 | 1.5 | −3.8 | 2.0 | 0.518 |
| | | VISIT2 | # | 30 | 18.6 | 4.9 | 17.0 | 9.2 | 29.6 | 31 | 17.5 | 5.8 | 17.0 | 6.6 | 29.6 | 1.6 | 1.1 | −0.5 | 3.7 | 0.124 |
| | | VISIT3 | # | 30 | 19.2 | 5.1 | 18.6 | 8.2 | 29.6 | 31 | 18.6 | 5.4 | 18.2 | 8.4 | 29.6 | 1.1 | 1.1 | −1.0 | 3.2 | 0.286 |
| | | Change1 | # | 30 | 1.6 | 4.2 | 1.2 | −7.0 | 9.4 | 31 | −0.4 | 5.0 | 0.0 | −20.8 | 7.4 | 1.6 | 1.1 | −0.5 | 3.7 | 0.124 |
| | | Change2 | # | 30 | 2.2 | 5.0 | 1.8 | −7.0 | 15.6 | 31 | 0.7 | 4.2 | 0.0 | −8.0 | 14.0 | 1.1 | 1.1 | −1.0 | 3.2 | 0.286 |
| [symbol] | score | VISIT1 | * | 30 | 22.3 | 6.1 | 23.3 | 11.5 | 29.5 | 31 | 23.1 | 7.0 | 25.5 | 7.0 | 29.5 | −0.8 | 1.7 | −4.1 | 2.6 | 0.644 |
| | | VISIT2 | # | 30 | 22.8 | 5.4 | 23.3 | 12.5 | 29.5 | 31 | 21.8 | 6.2 | 23.0 | 11.0 | 29.5 | 1.4 | 1.1 | −0.7 | 3.6 | 0.194 |
| | | VISIT3 | # | 30 | 22.7 | 6.0 | 22.0 | 11.5 | 29.5 | 31 | 22.8 | 5.9 | 23.5 | 11.5 | 29.5 | 0.3 | 1.1 | −1.9 | 2.5 | 0.761 |
| | | Change1 | # | 30 | 0.5 | 5.3 | 0.0 | −13.0 | 12.5 | 31 | −1.2 | 4.3 | 0.0 | −12.5 | 6.0 | 1.4 | 1.1 | −0.7 | 3.6 | 0.194 |
| | | Change2 | # | 30 | 0.4 | 4.6 | 0.0 | −11.0 | 10.0 | 31 | −0.3 | 5.5 | 0.0 | −13.0 | 13.0 | 0.3 | 1.1 | −1.9 | 2.5 | 0.761 |
| [symbol] | score | VISIT1 | * | 30 | 19.7 | 5.2 | 19.5 | 12.0 | 32.7 | 31 | 20.6 | 6.6 | 20.0 | 12.0 | 32.7 | −0.9 | 1.5 | −4.0 | 2.1 | 0.547 |
| | | VISIT2 | # | 30 | 20.3 | 4.9 | 20.2 | 12.0 | 32.7 | 31 | 21.6 | 6.4 | 21.7 | 11.7 | 32.7 | −0.7 | 1.1 | −2.8 | 1.5 | 0.551 |
| | | VISIT3 | # | 30 | 20.9 | 4.8 | 21.4 | 12.0 | 29.0 | 31 | 20.9 | 6.6 | 19.7 | 11.7 | 32.7 | 0.5 | 1.1 | −1.7 | 2.7 | 0.646 |
| | | Change1 | # | 30 | 0.7 | 3.7 | 0.0 | −5.6 | 9.7 | 31 | 1.0 | 5.3 | 1.0 | −17.4 | 12.0 | −0.7 | 1.1 | −2.8 | 1.5 | 0.551 |
| | | Change2 | # | 30 | 1.2 | 4.5 | 0.5 | −5.6 | 15.7 | 31 | 0.3 | 5.3 | 0.0 | −10.0 | 15.3 | 0.5 | 1.1 | −1.7 | 2.7 | 0.646 |
| [symbol] | score | VISIT1 | * | 30 | 19.8 | 4.4 | 20.5 | 10.5 | 28.0 | 31 | 20.7 | 5.9 | 21.5 | 5.5 | 31.5 | −1.0 | 1.3 | −3.6 | 1.7 | 0.474 |
| | | VISIT2 | # | 30 | 19.7 | 4.0 | 19.5 | 10.5 | 28.0 | 31 | 19.0 | 6.4 | 18.0 | 6.0 | 33.5 | 1.2 | 1.2 | −1.3 | 3.6 | 0.339 |
| | | VISIT3 | # | 30 | 20.0 | 4.6 | 19.8 | 8.5 | 28.0 | 31 | 18.8 | 6.4 | 18.0 | 8.0 | 33.5 | 1.7 | 1.2 | −0.7 | 4.2 | 0.169 |
| | | Change1 | # | 30 | −0.1 | 4.2 | 0.0 | −10.0 | 7.0 | 31 | −1.7 | 5.8 | −2.0 | −13.5 | 8.5 | 1.2 | 1.2 | −1.3 | 3.6 | 0.339 |
| | | Change2 | # | 30 | 0.3 | 4.5 | 0.0 | −10.0 | 14.5 | 31 | −2.0 | 6.5 | −3.5 | −12.0 | 14.0 | 1.7 | 1.2 | −0.7 | 4.2 | 0.169 |

[symbol]

indicates data missing or illegible when filed

As described above, according to this embodiment, a novel composition for increasing motivation and/or a novel composition for improving wakefulness upon waking up could be provided.

1. A method for increasing motivation after sleep, comprising administering to a human in need thereof a therapeutically or prophylactically effective amount of a composition comprising:
a partially hydrolyzed guar gum having an average molecular weight of 1.0×10³ to 2.0×10⁵,
wherein:
70% or more by mass of the components of the partially hydrolyzed guar gum fall within the range of 1.0×10³ to 2.0×10⁵,
a 1% by mass aqueous solution of the partially hydrolyzed guar gum has a viscosity, measured at 25° C. and 60 rpm using a B-type viscometer, of 50 mPa·s or less,
the partially hydrolyzed guar gum is produced by hydrolyzing galactomannan polysaccharide, which is obtained from guar endosperm and has a galactose to mannose content ratio (galactose:mannose) in the range of 1:1.3 to 1:2.1, using β-mannanase to reduce the molecular weight, and contains at least 70% by mass of dietary fiber as determined by an enzymatic HPLC method.

2. A method for improving wakefulness upon waking, comprising administering to a human in need thereof a therapeutically or prophylactically effective amount of a composition comprising:
a partially hydrolyzed guar gum having an average molecular weight of 1.0×10³ to 2.0×10⁵,
wherein:
70% or more by mass of the components of the partially hydrolyzed guar gum fall within the range of 1.0×10³ to 2.0×10⁵,
a 1% by mass aqueous solution of the partially hydrolyzed guar gum has a viscosity, measured at 25° C. and 60 rpm using a B-type viscometer, of 50 mPa·s or less,
the partially hydrolyzed guar gum is produced by hydrolyzing galactomannan polysaccharide, which is obtained from guar endosperm and has a galactose to mannose content ratio (galactose:mannose) in the range of 1:1.3 to 1:2.1, using β-mannanase to reduce the molecular weight, and contains at least 70% by mass of dietary fiber as determined by an enzymatic HPLC method.

3. The method according to claim 1, wherein the human is a healthy subject not having a sleep disorder.

4. The method according to claim 1, wherein the composition is contained in a food and drink product.

5. The method according to claim 1, wherein the composition is contained in a pharmaceutical product.

6. The method according to claim 2, wherein the human is a healthy subject not having a sleep disorder.

7. The method according to claim 2, wherein the composition is contained in a food and drink product.

8. The method according to claim 2, wherein the composition is contained in a pharmaceutical product.

9. The method of claim 2, wherein the method is for improving a feeling of being refreshed upon waking up or a feeling of reduced fatigue upon waking up.

10. The method of claim 1, wherein the therapeutically or prophylactically effective amount of the partially hydrolyzed guar gum is in the range of from 0.5 g to 70 g per day.

11. The method of claim 1, wherein the therapeutically or prophylactically effective amount of the partially hydrolyzed guar gum is in the range of from 3 g to 30 g per day.

12. The method of claim 1, wherein the therapeutically or prophylactically effective amount of the partially hydrolyzed guar gum is in the range of from 3 g to 18 g per day.

13. The method of claim 1, wherein 80% by mass or more of the components of the partially hydrolyzed guar gum have a molecular weight in a range from 1.0×10³ to 2.0×10⁵.

14. The method of claim 1, wherein the partially hydrolyzed guar gum has a molecular weight in a range from 2.0×10³ to 1.0×10⁵.

15. The method of claim 1, wherein the partially hydrolyzed guar gum has a molecular weight in a range from 2.0×10³ to 2.5×10⁴.

16. The method of claim 1, wherein the partially hydrolyzed guar gum is administered orally.

17. The method of claim 16, wherein the partially hydrolyzed guar gum has a molecular weight in a range from 2.0×10³ to 1.0×10⁵.

18. The method of claim 17, wherein the partially hydrolyzed guar gum has a molecular weight in a range from 2.0×10³ to 2.5×10⁴.

19. The method of claim 18, wherein 80% by mass or more of the components of the partially hydrolyzed guar gum have a molecular weight in a range from 1.0×10³ to 2.0×10⁵.

20. The method of claim 19, wherein the therapeutically or prophylactically effective amount of the partially hydrolyzed guar gum is in the range of from 3 g to 30 g per day.
